(19) United States
(12) Patent Application Publication
(10) Pub. No.: US 2023/0058342 A1
(43) Pub. Date: Feb. 23, 2023

(54) PHYSIOLOGICAL MONITORING CHAIR

(71) Applicant: MASIMO CORPORATION, Irvine, CA (US)

(72) Inventors: Massi Joe E. Kiani, Laguna Niguel, CA (US); Bilal Muhsin, Irvine, CA (US)

(21) Appl. No.: 17/820,981

(22) Filed: Aug. 19, 2022

Related U.S. Application Data
(60) Provisional application No. 63/235,609, filed on Aug. 20, 2021.

(51) Int. Cl. A61B 5/00; A61B 5/0205

(52) U.S. Cl. CPC A61B 5/6891; A61B 5/0205; A61B 5/7425; A61B 5/7455; A61B 5/4836; A61B 5/021

(57) ABSTRACT

Various chairs are described herein for monitoring physiological parameters of a subject. In some implementations, a physiological monitoring chair includes a base portion configured to support a weight of the patient, a back portion configured to at least partially support a back of the patient, a leg portion configured to be positioned adjacent legs of the patient, a vibration motor, at least one physiological measurement device usable for determining said one or more physiological parameters of the patient, and a controller. The controller can be configured to instruct the vibration motor to cause vibration of at least a portion of the physiological monitoring chair based on said determined one or more physiological parameters of the patient. In some implementations, one or both of the back and leg portions are configured to be moved between an upright position and a reclined position.

### RELATED APPLICATIONS

This application claims the benefit of U.S. Provisional Application No. 63/235,609, filed Aug. 20, 2021, entitled “Patient Chair With Integrated Physiological Measurement,” which is incorporated by reference herein in its entirety.

### TECHNICAL FIELD

The present disclosure relates to devices, methods, and/or systems for monitoring a subject's physiological information. More specifically, the present disclosure describes, among other things, chairs for monitoring and/or measuring one or more physiological parameters of a subject, and in some implementations, cause vibration of the subject's body.

### BACKGROUND

Hospitals, nursing homes, and other patient care facilities typically utilize a number of sensors, devices, and/or monitors to collect or analyze a patient's physiological parameters such as blood oxygen saturation level, temperature, respiratory rate, pulse rate, blood pressure, and the like. Such devices can include, for example, acoustic sensors, electroencephalogram (EEG) sensors, electrocardiogram (ECG) devices, blood pressure monitors, temperature sensors, pulse oximeters, among others. In medical environments, various sensors/devices (such as those just mentioned) are attached to a patient and connected to one or more patient monitoring devices using cables or via wireless connection. Patient monitoring devices generally include sensors, processing equipment, and displays for obtaining and analyzing a medical patient's physiological parameters. Clinicians, including doctors, nurses, and other medical personnel, use the physiological parameters obtained from patient monitors to diagnose illnesses and to prescribe treatments. Clinicians also use the physiological parameters to monitor patients during various clinical situations to determine whether to increase the level of medical care given to patients.

### SUMMARY

In care facilities (such as hospitals) patients are often seated/laying in hospital beds/chairs and a variety of physiological measurement devices are attached to the patients' body for physiological monitoring. Such devices are separate from the hospital beds/chairs and many include cables for communicating with other devices and/or for receiving power. Such devices often send (via wired or wireless means) physiological data to other devices (positioned away from the patients and the beds/chairs) for processing and/or display. Accordingly, in such scenarios, the hospital bed/chair is separate and distinct from the physiological measurement devices and external processing/display devices and no integration (or interaction) exists between the chair and these devices. It would be beneficial for a bed/chair in a care facility, and outside care facilities (such as in a home or other environment), to include and/or interact with physiological measurement devices.

The present disclosure describes various implementations of chairs that can include and/or interact with a variety of devices for the purpose of monitoring physiological parameters and/or condition of a subject. Some implementations of the chairs include one or more physiological measurement devices (which also may be referred to herein as “physiological monitoring devices” or “physiological sensors”), for example, embedded into portion(s) of the chair and/or permanently or detachably connected to portion(s) of the chair via cables. Some implementations of the chairs described herein are configured to transition from and/or between various positions, for example, upright, reclined, and/or flat positions. Some implementations of the chairs described herein are configured to transition from and/or between such positions based on values of one or more physiological parameters of the subject seated/laying in the chairs (for example, when/if a given physiological parameter exceeds or falls below a threshold). Some implementations of the chairs described herein include vibration motor(s) configured to vibrate portions of the chair, for example, based on values of one or more physiological parameters of the subject seated/laying in the chairs (for example, when/if a given physiological parameter exceeds or falls below a threshold). Some implementations of the chairs described herein include one or more displays embedded into portion(s) of the chair and/or include a display that is mounted away from portions of the chair (for example, with one or more mounting arms), and such displays can present one or more physiological parameters, information related to such parameters, and/or other information. Some implementations of the chairs described herein include cable management features that reduce the encumbrance caused by cables connected to physiological measurement devices. For example, some implementations of the chairs described herein include one or more hooks on various portions of the chair that can support portions of cables and/or include one or more connection ports on various portions of the chair so as to minimize lengths of cables of physiological measurement devices connected to the chair.

Although the term “chair” is used throughout the disclosure, such term is not intended to be limiting. Various implementations of the chairs disclosed herein can be configured to be used in various positions, and in some cases, can be utilized as a bed. Further, any of the chairs disclosed herein can be utilized in any setting or environment, including but not limited to a care facility (such as a hospital) among other settings or environments. For example, any of the chairs disclosed herein can be utilized in a subject's home. Indeed, in medical and health monitoring, outside clinical environments are becoming increasingly more prevalent and this is at least partially due to greater interest and need for health monitoring of persons on a continual basis during daily routines and activities and inside the home.

Disclosed herein is a physiological monitoring chair for monitoring one or more physiological parameters of a subject. In some implementations, the physiological monitoring chair comprises: a base portion configured to rest atop a surface and support a weight of the subject; a back portion connected to the base portion and configured to at least partially support a back of the subject when the subject is seated in the physiological monitoring chair; a leg portion connected to the base portion and configured to be positioned adjacent to legs of the subject when the subject is seated in the physiological monitoring chair; at least one vibration motor; and a controller in communication with the at least one vibration motor, wherein the controller is configured to receive physiological data indicative of one or more physiological parameters of the subject from at least one physiological measurement device and instruct the at least one vibration motor to cause vibration of at least a portion of the physiological monitoring chair based on said one or more physiological parameters of the subject.

In some implementations, the physiological monitoring chair includes said at least one physiological measurement device. In some implementations, said at least one physiological measurement device includes an inflatable cuff configured to wrap around a portion of the subject's arm such that the inflatable cuff is pneumatically coupled to a portion of the chair via a tube and the controller is configured to determine blood pressure data when the inflatable cuff is in use. In some implementations, the physiological monitoring chair further includes a hook configured to hold a portion of said tube coupled to said inflatable cuff. In some implementations, the physiological monitoring chair includes at least one armrest portion connected to the base portion and configured to support an arm of the subject when the subject is seated in the physiological monitoring chair.

In some implementations, the at least one physiological measurement device includes at least one of an oximetry sensor, a temperature sensor, and/or an electroencephalography (EEG) sensor. In some implementations, said at least one physiological measurement device is integrated into the armrest portion. In some implementations, at least a portion of the armrest portion includes a flat surface. In some implementations, the physiological monitoring chair includes an at least partially transparent window on the armrest portion and said at least one physiological measurement device is positioned underneath the window. In some implementations, said at least one physiological measurement device includes an oximetry sensor. In some implementations, said at least one physiological measurement device includes an electrocardiogram (ECG) sensor. In some implementations, said at least one physiological measurement device includes an electroencephalography (EEG) sensor. In some implementations, said at least one physiological measurement device includes a capnograph. In some implementations, said at least one physiological measurement device includes a temperature sensor. In some implementations, said at least one physiological measurement device includes an acoustic sensor.

In some implementations, said at least one physiological measurement device includes a blood pressure monitor. In some implementations, said at least one physiological measurement device includes an inflatable cuff configured to wrap around a portion of the subject's arm, and the inflatable cuff is within a housing. In some implementations, the housing is a rigid structure. In some implementations, the chair includes at least one mounting arm connected to a portion of the chair, wherein the housing is coupled to the mounting arm. In some implementations, the at least one mounting arm includes a first mounting arm connected to the portion of the chair and a second mounting arm movably connected to the first mounting arm, the housing is coupled to the second mounting arm, and the first mounting arm, second mounting arm, and housing is configured to allow a position of the housing to be moved. In some implementations, said at least one physiological measurement device includes an inflatable cuff configured to wrap around a portion of the subject's arm, the inflatable cuff is pneumatically coupled to a blood pressure monitor, the blood pressure monitor is configured to determine blood pressure data when the inflatable cuff is in use, and the controller receives blood pressure values from the blood pressure monitor. In some implementations, the physiological monitoring chair also includes one or more connection ports and wherein a cable connects the blood pressure monitor to one or more connection ports such that the controller receives the blood pressure values from the blood pressure monitor via the cable and the connection port. In some implementations, the leg portion includes a cavity configured to receive and surround a portion of the legs of the subject when the subject is seating in the physiological monitoring chair.

In some implementations, the physiological monitoring chair includes a display configured to display information relating to said one or more physiological parameters. In some implementations, said display is located on a side of the back portion that does not face toward the patient when the patient is seated in the physiological monitoring chair. In some implementations, said display is coupled to a mounting assembly configured to mount the display at a location away from the chair. In some implementations, the mounting assembly is coupled to the base portion. In some implementations, the controller is configured to wirelessly receive physiological information from the at least one physiological measurement device. In some implementations, the physiological monitoring chair is configured to provide power for said at least one physiological measurement device. In some implementations, the physiological monitoring chair also includes one or more connection ports and wherein the at least one physiological measurement device includes a cable configured to connect to and receive power from one of the one or more connection ports of the physiological monitoring chair. In some implementations, the physiological monitoring chair is configured to receive power from an external power source via a cable. In some implementations, responsive to receiving said physiological data indicative of lower torso edema of said subject from the at least one physiological measurement device, the controller is configured to instruct the at least one vibration motor to cause vibration of the leg portion.

Disclosed herein is a physiological monitoring chair for monitoring one or more physiological parameters of a subject In some implementations, the physiological monitoring chair comprises: a base portion configured to rest atop a surface and support a weight of the subject; a back portion connected to the base portion and configured to at least partially support a back of the subject when the subject is seated in the physiological monitoring chair; a leg portion connected to the base portion and configured to be positioned adjacent to legs of the subject when the subject is seated in the physiological monitoring chair; a controller in communication with said motor, the controller configured to receive physiological data indicative of one or more physiological parameters of the subject from at least one physiological measurement device and cause the physiological monitoring chair to transition from a first configuration to a second configuration based on said one or more physiological parameters of the subject, wherein, when the physiological monitoring chair is in said first configuration, at least one of said back portion and said leg portion is oriented non-parallel with respect to said surface, and wherein when the physiological monitoring chair is in said second configuration, said back portion and said leg portion are oriented substantially parallel with respect to said surface.

In some implementations, the physiological monitoring chair also includes a motor and one or more struts coupled to said motor and to one or both of said back portion and said leg portion, wherein said controller is configured to cause the physiological monitoring chair to transition from the first configuration to the second configuration by instructing the motor to cause movement of the one or more struts. In some implementations, the physiological monitoring also includes said at least one physiological measurement device. In some implementations, said at least one physiological measurement device includes an inflatable cuff configured to wrap around a portion of the subject's arm, the inflatable cuff is pneumatically coupled to a portion of the chair via a tube, and the controller is configured to determine blood pressure data when the inflatable cuff is in use. In some implementations, the physiological monitoring chair also includes a hook configured to hold a portion of said tube coupled to said inflatable cuff. In some implementations, the physiological monitoring chair also includes at least one armrest portion connected to the base portion and configured to support an arm of the subject when the subject is seated in the physiological monitoring chair.

In some implementations, said at least one physiological measurement device includes an oximetry sensor. In some implementations, said at least one physiological measurement device includes an electrocardiogram (ECG) sensor. In some implementations, said at least one physiological measurement device includes an electroencephalography (EEG) sensor. In some implementations, the physiological monitoring chair also includes an at least partially transparent window on the armrest portion and said at least one physiological measurement device is positioned underneath the window. In some implementations, said at least one physiological measurement device includes an oximetry sensor. In some implementations, said at least one physiological measurement device includes an electrocardiogram (ECG) sensor. In some implementations, said at least one physiological measurement device includes an electroencephalography (EEG) sensor. In some implementations, said at least one physiological measurement device includes a capnograph. In some implementations, said at least one physiological measurement device includes a temperature sensor. In some implementations, said at least one physiological measurement device includes an acoustic sensor.

In some implementations, said at least one physiological measurement device includes a blood pressure monitor. In some implementations, said at least one physiological measurement device includes an inflatable cuff configured to wrap around a portion of the subject's arm and wherein the inflatable cuff is within a housing. In some implementations, the housing includes a rigid structure. In some implementations, the physiological monitoring chair also includes at least one mounting arm connected to a portion of the chair, wherein the housing is coupled to the mounting arm. In some implementations, the at least one mounting arm includes a first mounting arm connected to the portion of the chair and a second mounting arm movably connected to the first mounting arm, the housing is coupled to the second mounting arm, and the first mounting arm, second mounting arm, and housing is configured to allow a position of the housing to be moved. In some implementations, said at least one physiological measurement device includes an inflatable cuff configured to wrap around a portion of the subject's arm; and the inflatable cuff is pneumatically coupled to a blood pressure monitor, the blood pressure monitor is configured to determine blood pressure data when the inflatable cuff is in use; the controller receives blood pressure values from the blood pressure monitor. In some implementations, the physiological monitoring chair also includes one or more connection ports and wherein a cable connects the blood pressure monitor to one or more connection ports such that the controller receives the blood pressure values from the blood pressure monitor via the cable and the connection port. In some implementations, the leg portion includes a cavity configured to receive and surround a portion of the legs of the subject when the subject is seating in the physiological monitoring chair.

In some implementations, the physiological monitoring chair also includes a display configured to display information relating to said one or more physiological parameters. In some implementations, said display is located on a side of the back portion that does not face toward the patient when the patient is seated in the physiological monitoring chair. In some implementations, said display is coupled to a mounting assembly configured to mount the display at a location away from the chair. In some implementations, the mounting assembly is coupled to the base portion. In some implementations, the controller is configured to wirelessly receive physiological information from the at least one physiological measurement device.

In some implementations, the physiological monitoring chair is configured to receive power from an external power source via a cable. In some implementations, the physiological monitoring chair is configured to provide power for said at least one physiological measurement device. In some implementations, the physiological monitoring chair also includes one or more connection ports and wherein each of the at least one physiological measurement device includes a cable configured to connect to and receive power from one of the one or more connection ports of the physiological monitoring chair.

Disclosed herein is a physiological monitoring chair for monitoring one or more physiological parameters of a subject. In some implementations, the physiological monitoring chair comprises: a base portion configured to rest atop a surface and support a weight of the subject; a back portion connected to the base portion and configured to at least partially support a back of the subject when the subject is seated in the physiological monitoring chair; a leg portion connected to the base portion and configured to be positioned adjacent to legs of the subject when the subject is seated in the physiological monitoring chair; a first armrest portion connected to the base portion and configured to support an arm of the subject when the subject is seated in the physiological monitoring chair; and an oximetry sensor operably positioned within the first armrest portion, the oximetry sensor comprising at least one emitter configured to emit light into tissue of the subject when in use and at least one detector configured to detect at least a portion of the emitted light after passing through a portion of said tissue when in use.

In some implementations, the oximetry sensor includes a plurality of emitters and a plurality of detectors. In some implementations, the at least one emitter and at least one detector are arranged in a reflectance configuration. In some implementations, said oximetry sensor is operably positioned on flat portion of said armrest portion. In some implementations, the physiological monitoring chair also includes an at least partially transparent window along a portion of the armrest portion overtop the oximetry sensor. In some implementations, the physiological monitoring chair also includes: a second armrest portion connected to the base portion and configured to support another arm of the subject when the subject is seated in the physiological monitoring chair; and an additional oximetry sensor operably positioned within the second armrest portion, the oximetry sensor comprising at least one emitter configured to emit light into tissue of the subject when in use and at least one detector configured to detect at least a portion of the emitted light after passing through a portion of said tissue when in use. In some implementations, the physiological monitoring chair also includes: a second armrest portion connected to the base portion and configured to support another arm of the subject when the subject is seated in the physiological monitoring chair; a first ECG electrode operably positioned within the first armrest portion and a second ECG electrode operably positioned within the second armrest portion; a controller configured to receive one or more signals from said first and second ECG electrodes responsive to cardiac activity of the subject.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

### BRIEF DESCRIPTION OF THE DRAWINGS

Certain features of this disclosure are described below with reference to the drawings. The illustrated embodiments are intended to illustrate, but not to limit, the embodiments. Various features of the different disclosed embodiments can be combined to form further embodiments, which are part of this disclosure.

FIG. 1A illustrates a front perspective view of a physiological monitoring chair in accordance with aspects of this disclosure.

FIG. 1B illustrates the physiological monitoring chair of FIG. 1A with a subject seated in the chair and the chair in a reclined position in accordance with aspects of this disclosure.

FIG. 1C illustrates the physiological monitoring chair of FIG. 1A with a subject seated in the chair and the chair in a flat position in accordance with aspects of this disclosure.

FIG. 1D illustrates a back perspective view of the physiological monitoring chair of FIG. 1A in accordance with aspects of this disclosure.

FIG. 1E illustrates a schematic representation of physiological measurement module of the physiological monitoring chair of FIG. 1A in accordance with aspects of this disclosure.

FIG. 1F illustrates the physiological monitoring chair of FIG. 1A with a subject seated in the chair and legs of the subject received into cavities of another implementation of a leg portion in accordance with aspects of this disclosure.

FIG. 1G illustrates an implementation of a display of the physiological monitoring chair of FIG. 1A in accordance with aspects of this disclosure.

FIG. 1H illustrates another implementation of a display of the physiological monitoring chair of FIG. 1A in accordance with aspects of this disclosure.

FIG. 2A illustrates a front perspective view of another implementation of a physiological monitoring chair in accordance with aspects of this disclosure.

FIG. 2B illustrates a back perspective view of the physiological monitoring chair of FIG. 2A in accordance with aspects of this disclosure.

FIG. 3 illustrates a front perspective view of another implementation of a physiological monitoring chair in accordance with aspects of this disclosure.

FIGS. 4A-4D illustrate another implementation of a physiological monitoring chair and various physiological measurement devices that can be included in the chair and/or can interact with the chair in accordance with aspects of this disclosure.

FIG. 5 illustrates a schematic diagram of certain features that can be included in any of the physiological monitoring chairs described herein in accordance with aspects of this disclosure.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed embodiments and/or uses and obvious modifications and equivalents thereof. Thus, it is intended that the scope of this disclosure should not be limited by any particular embodiments described below. The features of the illustrated embodiments can be modified, combined, removed, and/or substituted as will be apparent to those of ordinary skill in the art upon consideration of the principles disclosed herein.

Various implementations of physiological monitoring chairs are disclosed herein that can be utilized for physiological monitoring of a subject. The physiological monitoring chairs can include and/or can interact with one or more physiological measurement devices that can be utilized to measure one or more physiological parameters of subjects, for example, when positioned in the chairs. Such one or more physiological measurement devices can be connected to portion(s) of the chairs via cables which can be integral with portions of the chairs and/or can removably connect to portion(s) of the chairs (for example, via a connection port of the chairs) as described in more detail below. Additionally or alternatively, some implementations of the physiological monitoring chairs disclosed herein can communicate with (for example, wirelessly communicate) with separate physiological measurement devices when a subject is seated in the chairs and/or when the subject is not seated in the chairs. In various implementations, the disclosed physiological monitoring chairs can include various structure as further described below (for example, base portion, back portion, leg portion, armrest portion(s), among others). In various implementations, the physiological monitoring chairs can be configured to transition from and/or between various configurations, for example, by moving portions of the chairs. For example, where the physiological monitoring chairs include back, base, leg, and/or armrest portions, the physiological monitoring chairs (for example, any of the back, base, leg, and/or armrest portions) can be configured to transition from an upright position to a reclined position, in a manner that can be similar to recliner chairs. Some implementations of the physiological monitoring chairs disclosed herein can be transitioned to a flat position that mimics a flat bed. As discussed in more detail below, in various implementations, the physiological monitoring chairs can include one or more vibration motors that can cause vibration of various portions of the chairs and thus, in turn, various portions of a subject's body when the subject is seated in the chairs. For example, the physiological monitoring chairs can include one or more vibration motors that can cause vibration of a base portion, a back portion, a leg portion, and/or one or more armrest portions of the chairs. As described further below, in some implementations, such vibration is initiated and/or adjusted based on one or more physiological parameters determined by the physiological monitoring chairs and/or by physiological measurement devices connected to the chairs.

Although the phrase “physiological monitoring chair” is used throughout the present disclosure, use of this phrase is not intended to mean that all of the chairs described herein necessarily and/or directly “monitor” physiological parameters. In some implementations, the chairs described herein can be utilized for physiological monitoring purposes (along with other device(s)) but may not directly monitor (for example, may not store and/or process) physiological parameters themselves. For example, the present disclosure contemplates chairs that can be utilized as a “hub” to receive physiological data and/or to transmit (for example, wirelessly) such data to external devices (for example, devices associated with caregivers) for display and/or processing.

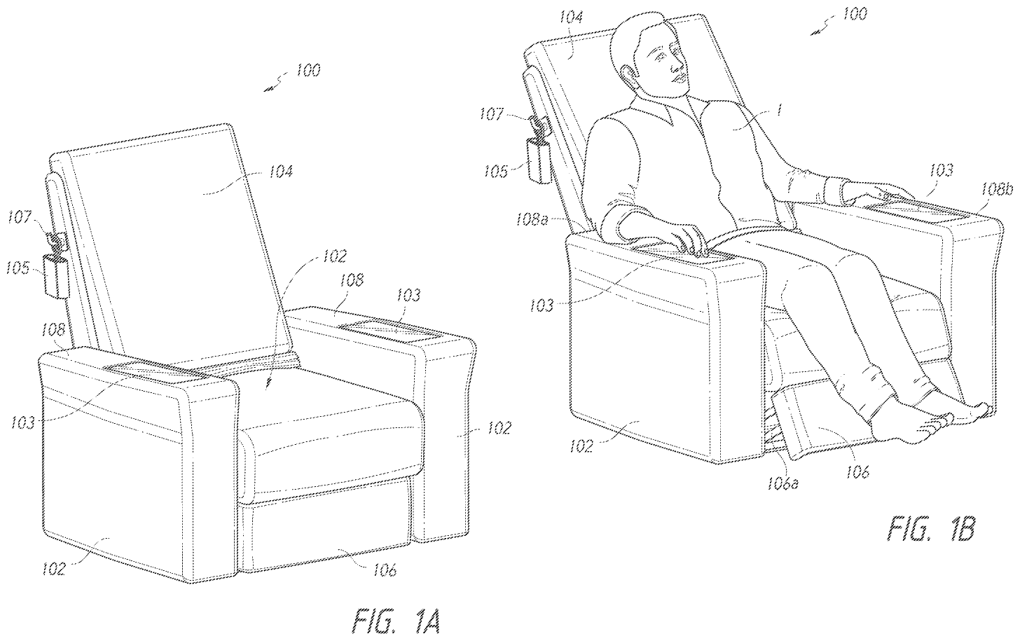

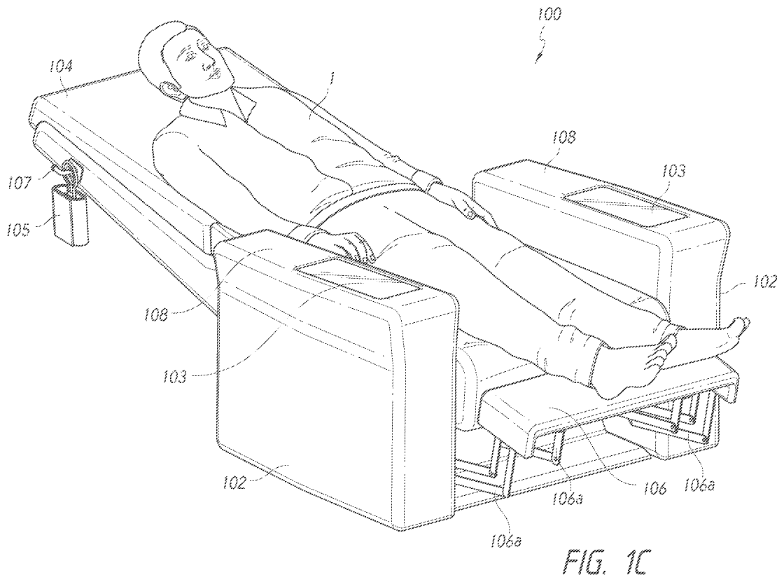

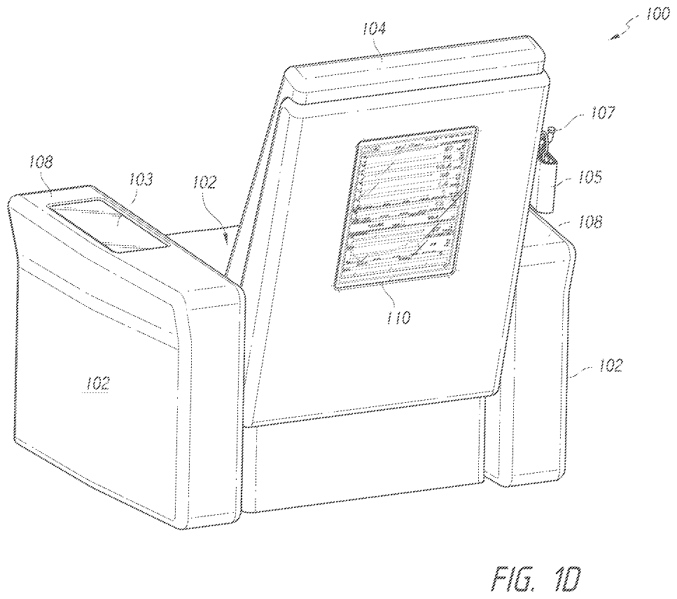

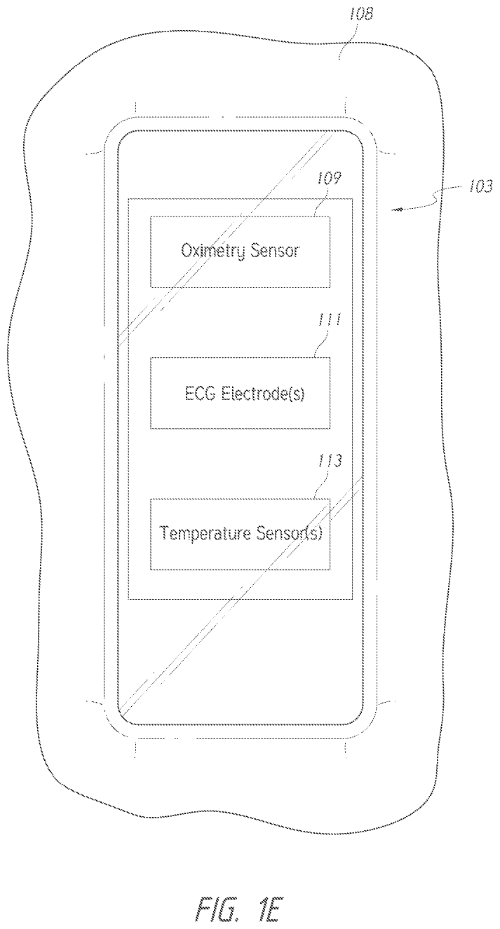

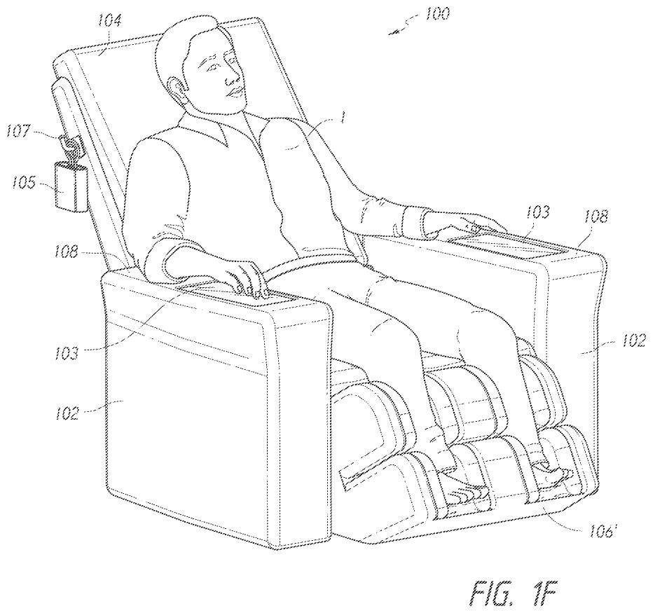

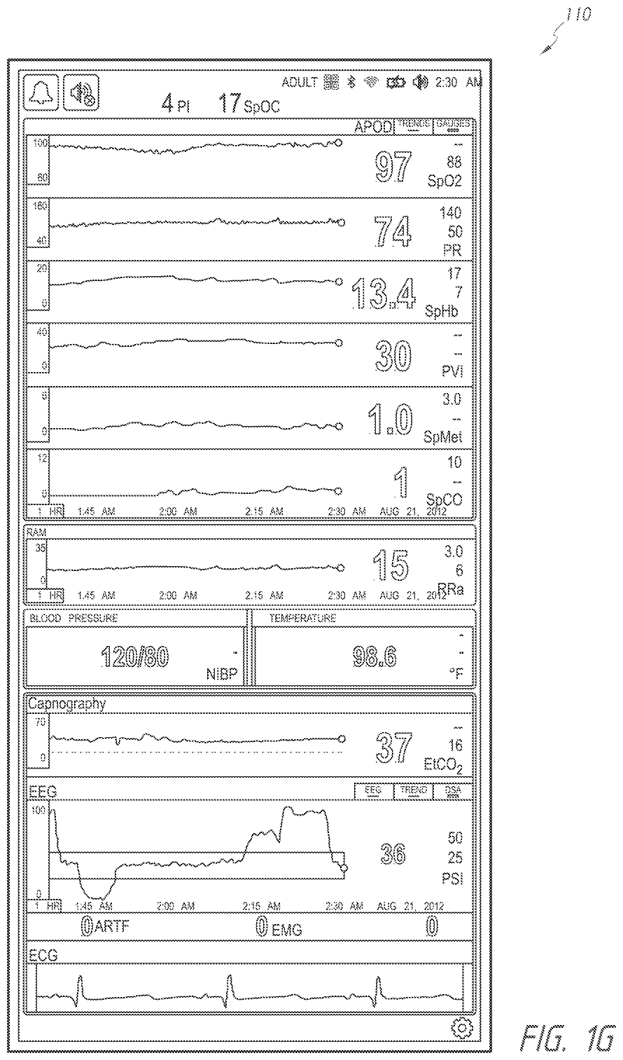

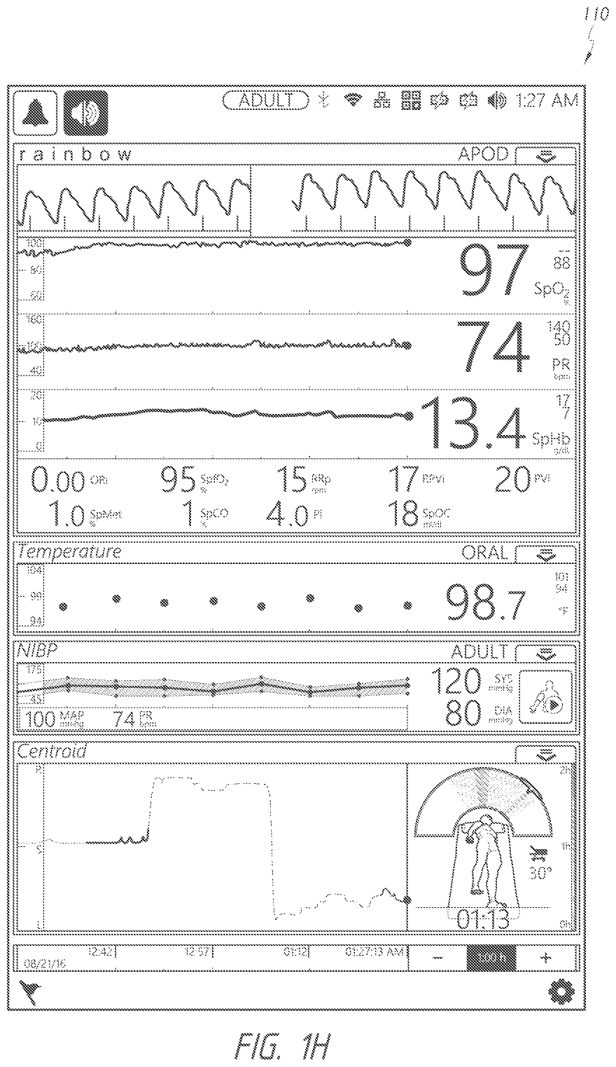

FIG. 1A illustrates a front perspective view of a physiological monitoring chair 100. Chair 100 can include a base portion 102 that can rest atop a surface (for example, ground or floor surface inside a home or clinical environment) and receive and support a weight of a subject 1. Chair 100 can include a back portion 104 that can be connected to the base portion 102 and can be configured to at least partially support a back of the subject 1 when seated and/or laying in the physiological monitoring chair 100. Physiological monitoring chair 100 can include a leg portion 106 connected to the base portion 102 and configured to be positioned adjacent legs of the subject 1 when seated in the chair 100. Chair 100 can include leg portion supports 106a coupled to the base portion 102 and the leg portion 106 such that leg portion 106 is extendable and/or moveable to transition leg portion 106 to different position (such as when the chair is in any of the positions discussed elsewhere herein). Chair 100 can include one or more armrest portions configured to support arms of the subject 1 when seated in chair 100. Such armrest portions can be integral with another portion of the chair 100 (such as with base portion 102 and/or with sidewalls that may form part of base portion 102) or such armrest portions can be separate and/or spaced from portions of the chair 100. With reference to FIGS. 1A-1B, chair 100 can include armrest portions 108. In some implementations, base portion 102 can include a seat portion and/or surface upon which subject 1 can sit. In some implementations, armrest portions 108 are on a top portion of sidewalls that form part of base portion 102, as shown in FIG. 1A. However, in some implementations, base portion 102 does not include such sidewalls, for example, and includes only a seat portion and/or surface upon which subject 1 can sit. In other implementations, armrest portion 108 can be separate or partially separate from base portion 102. Any of base portion 102, back portion 104, leg portion 106, and/or armrest portions 108 can include and/or be made of various materials. For example, any of base portion 102, back portion 104, leg portion 106, and/or armrest portions 108 can include and/or be made of a fabric material and/or can include cushion(s). Chair 100 can include one or more or a plurality of physiological measurement devices, such as any of those discussed herein.

In some implementations, chair 100 is configured to transition from and/or between a variety of positions, for example an upright position and a reclined position (for example, a flat position or a position that is between the upright position and such flat position). Such chair positions are also referred to herein as “configurations”. An example upright position can be that shown in FIG. 1A. In some implementations, when the chair 100 is in an upright position: the back portion 104 is substantially perpendicular to a ground surface upon which the chair 100 rests; a seat of base portion 102 is substantially parallel to such ground surface; and/or leg portion 106 is substantially perpendicular to such ground surface. Example reclined positions are illustrated in FIGS. 1B and 1C. FIG. 1C illustrates a flat position of chair 100, for example, where the back portion 104 and other portions of chair 100 (such as a seat of base portion 102 and/or leg portion 106) are substantially parallel with respect to a ground surface upon which chair 100 rests. In some implementations, when the chair 100 is in flat position, the back portion 104, a seat of base portion 102, and leg portion 106 are oriented along a common plane. In some implementations, when the chair 100 is in a flat position, it mimics a bed. In some implementations, when the chair 100 is in an upright position, the leg portion 106 is substantially perpendicular to a plane defined by a seat of base portion 102. In some implementations, chair 100 includes a motor and one or more struts 106a (see FIGS. 1B-1C) coupled to said motor and to one or both of said back portion 104 and said leg portion 106. In some implementations, a controller of chair 100 is configured to cause the chair 100 to transition from and/or between any of the positions described herein (for example, upright, flat, and/or a reclined position between the upright and flat positions) by instructing the motor to cause movement of the one or more struts 106a.

Chair 100 can be transitioned to a variety of reclined positions that are between any of the above-described upright and flat positions. In such reclined positions, an angle between a plane defined by a seat of base portion 102 and the leg portion 106 can be not equal to 90 degrees (for example, greater than 90 degrees) and/or less than 180 degrees. For example, chair 100 can be transitioned to a variety of reclined positions between the upright and flat positions where an angle between back portion 102 and a seat of base portion 102 is 95 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, or 175 degrees, or any value or range within or bounded by any of these values or ranges.

Chair 100 can include various mechanical and/or electronic structure that connects the back portion 104 and leg portion 106 to the base portion 102 so as to allow the chair 100 to be transitioned between any of the above-described positions. For example, chair 100 (for example, any portions thereof) can be transitioned from and/or between upright and any reclined position by a mechanical mechanism able to be moved (for example, pulled) by the subject 1 and/or by an electronic mechanism (for example, by utilizing a user input device instructing a controller of the chair 100 to instruct various electronic and/or mechanical mechanisms to move portions of the chair 100).

In some implementations, chair 100 (or any of the other chairs disclosed herein, such as chair 200, 300, 400, 500) is configured to transition from and/or between any of the above-described positions based on one or more physiological parameters. For example, a controller of chair 100 (which can be similar or identical to controller 502 described elsewhere herein) can be configured to determine one or more physiological parameters based on signal(s) received from one or more physiological sensors (such as any of those disclosed herein) or receive determined physiological parameters from such sensor(s), and, based on such parameters, cause the chair 100 to transition from a first position to a second position. In some implementations, such controller can receive and/or process physiological data obtained from the any of the physiological measurement devices described herein such as oxygen saturation (SpO2), pulse rate (PR), total hemoglobin (SpHb), pleth variability index (PVI), methemoglobin (SpMet), carboxyhemoglobin (SpCO), respiration rate (RRa), blood pressure (systolic and/or diastolic) (for example, noninvasive blood pressure (NIBP)), temperature (in Fahrenheit and/or Celsius), end-tidal cardon dioxide (EtCO₂), EEG data, and/or ECG data, among other types of physiological data. In some implementations, such controller of chair 100 can cause the chair 100 to transition from and/or between any of the chair positions described herein (e.g., for example, upright, flat, and/or reclined position between such upright and flat positions) when one or more of (for example, combinations of) any of such parameters exceed or fall below a threshold and/or are outside of a predetermined range (that may be associated with normal and/or average ranges). Such controller of chair 100 can cause the chair 100 to transition from and/or between any of the chair positions described herein based on physiological data received from any of the physiological measurement devices described herein which may be wearable devices configured to be attached to portions of a subject's body.

As mentioned above, chair 100 can include and/or can interact with one or more or a plurality of physiological measurement devices usable for determining one or more physiological parameters. Chair 100 can act as a hub that connects to (via wired and/or wireless means) one or more or a plurality of physiological measurement devices and/or to separate devices via wired and/or wireless means. For example, chair 100 can receive and process physiological data obtained from physiological measurement device(s), transmit (via wired and/or wireless means) such physiological data to separate devices, receive information and/or instructions from separate devices (for example, instructions to have one or more physiological measurement devices of the chair 100 to perform measurements), and/or can instruct one or more physiological measurement devices of the chair 100 to perform measurements. For example, chair 100 can be configured to operate in a manner similar to any of the patient monitors discussed in U.S. Pat. No. 10,383,527 and/or medical monitoring hubs discussed in U.S. Pat. No. 10,010,276. Each of U.S. Pat. Nos. 10,383,527 and 10,010,276 are incorporated by reference herein in their entireties and form part of the present disclosure. Physiological monitoring chair 100 can include any of the patient monitors discussed in U.S. Pat. No. 10,383,527 and/or medical monitoring hubs discussed in U.S. Pat. No. 10,010,276 integrated into the chair 100 and/or chair 100 can include features of any of such patient monitors and/or medical monitoring hubs integrated therewithin. This can advantageously allow the chair 100 to allow for physiological monitoring of a subject and/or communication with separate devices (for example, a caregiver monitoring station in a hospital) without the need for a separate patient monitor or hub that facilitates communication between one or more physiological measurement devices on the subject and such separate devices. This can be significantly beneficial in clinical settings in order to decrease clutter and objects in the vicinity of the patient that may interfere with caregivers' ability to care for the patient. Additionally, when used in a non-clinical environment (for example, within a home), chair 100 can allow for physiological monitoring (for example, in a continuous manner) in a comfortable and convenient manner.

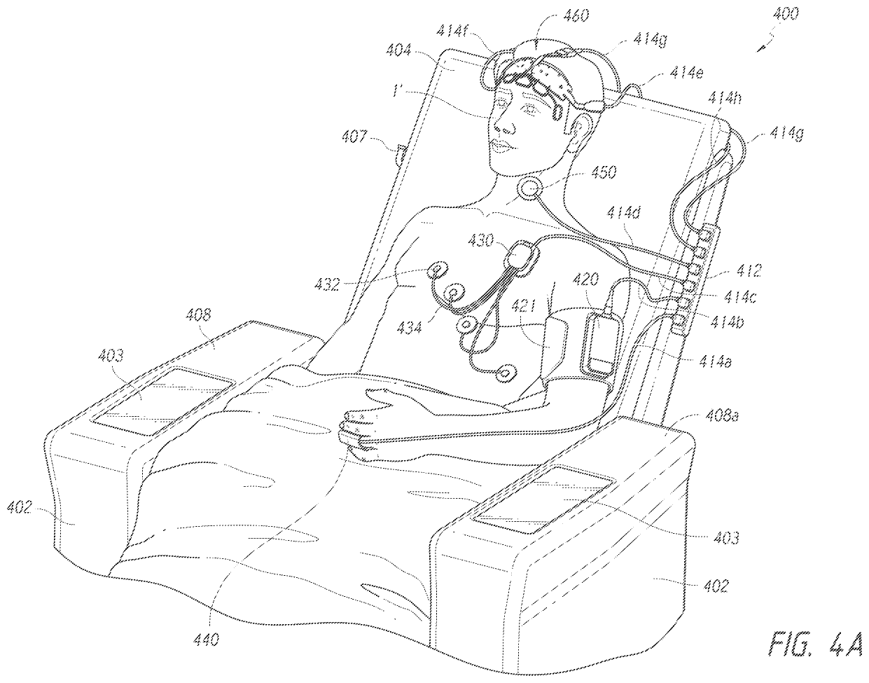

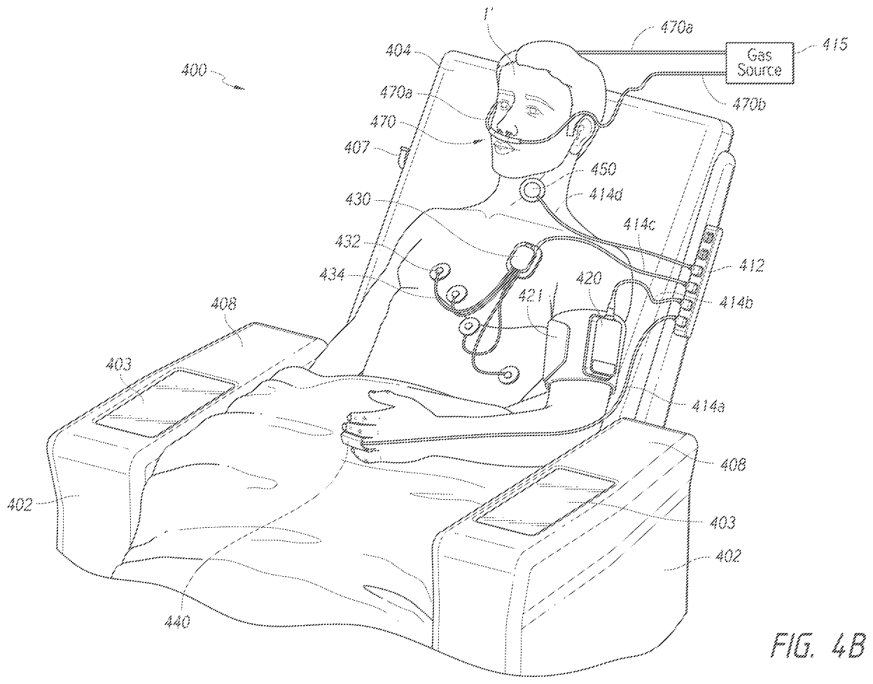

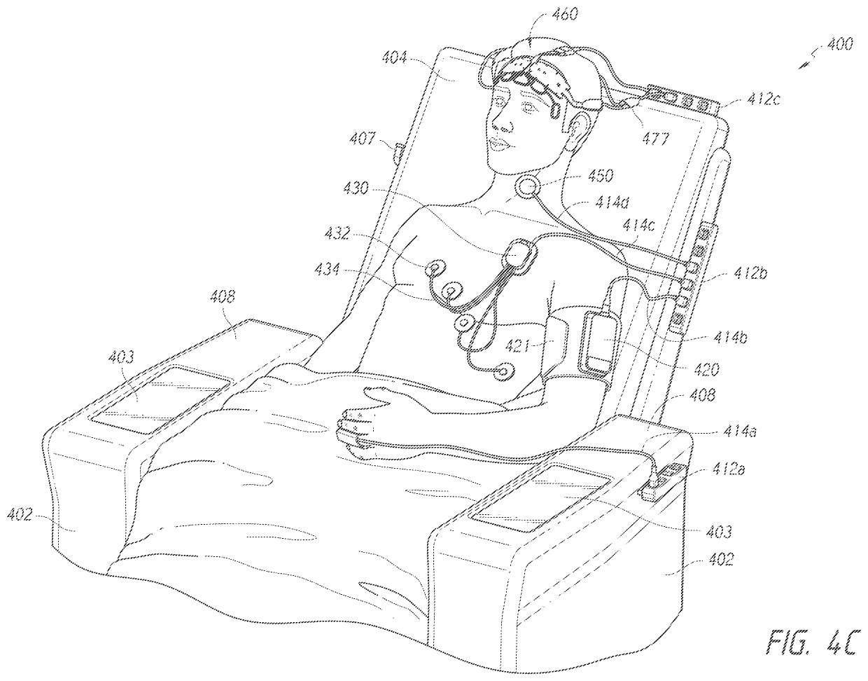

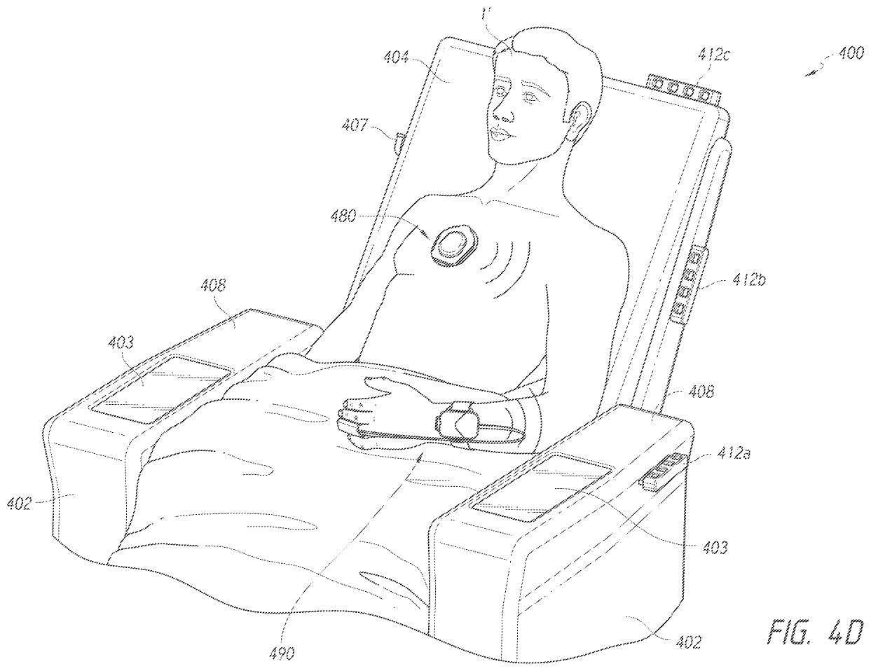

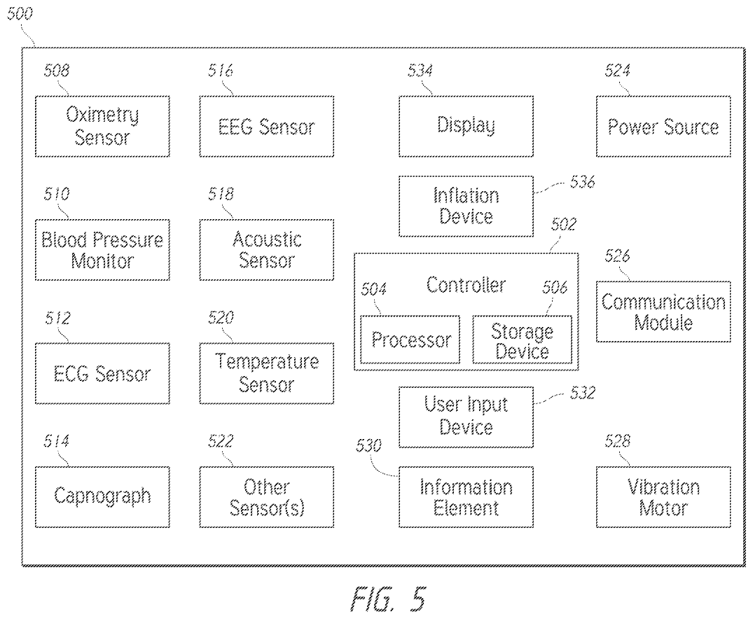

In some implementations, chair 100 includes a blood pressure monitor for monitoring blood pressure of a subject 1. Such blood pressure monitor can be a device that monitors and/or measures blood pressure of subject 1 and that is connected to a portion of the chair 100 (for example, an electronic module of the chair 100) for example, via a cable. Such blood pressure monitor can be similar or identical to any of the blood pressure monitors described in U.S. Publication No. 2020/0329983, which is incorporated by reference herein in its entirety and forms part of the present disclosure. For example, chair 100 can include a blood pressure monitor similar or identical to blood pressure monitor 420 (with connected cuff 421) illustrated in FIG. 4A, and such blood pressure monitor 420 can be permanently connected to chair 100 or removably connected to chair 100 (for example, via a connection port of chair 100 similar to connection port 412). In some implementations, chair 100 is connected to an inflatable cuff 105 via a tube (see FIGS. 1A-1B), and chair 100 (for example, a controller of chair 100). In such implementations, chair 100 can inflate cuff 105 (for example, with an inflation device of chair 100 that can be similar or identical to inflation device 536) and can determine blood pressure values (for example, systolic and/or diastolic blood pressure values) of subject 1 using cuff 105. Cuff 105 can be configured to wrap around a portion of a subject's arm. Cuff 105 can be pneumatically coupled to a portion of the chair 100 via a tube. In order to obtain a measurement, the cuff 105 can be secured around the patient's arm at the measurement site. Chair 100 (for example, via a controller of chair 100 and/or a blood pressure monitor integrated into chair 100) can then implement desired inflation and deflation profiles for cuff 105 to obtain an output signal which can be processed to yield one or more blood pressure measurement values. During this process, when the air pressure inside the cuff 105 is greater than the minimum, or diastolic blood pressure—but less than the maximum, or systolic blood pressure—inside the artery, the cuff 105 partially collapses the arterial wall at the measurement site. The partial collapse of the arterial wall restricts blood flow through the artery. The degree of collapse—and the resulting restriction of blood flow through the artery—depends on the extent to which the air pressure in the cuff 105 exceeds the minimum blood pressure in the artery. When the air pressure inside the cuff 105 rises to exceed the maximum blood pressure in the artery, the artery becomes occluded and blood flow is cut off. In some implementations, chair 100 includes a hook 107 configured to hold and/or support a portion of a tube coupled to the inflatable cuff 105. The hook 107 can be coupled to a portion of the chair and within reach of the subject 1, for example, the back portion 104. In some implementations, chair 100 includes a hook 107 on both sides of back portion 104 to facilitate use of cuff 105 on either the right or left arm of the subject 1.

Chair 100 can include one or more physiological measurement devices as discussed elsewhere herein. In some implementations, chair 100 includes one or more physiological measurement devices embedded into portions of the chair 100. With reference to FIGS. 1A-1D and IF, chair 100 can include one or more physiological sensors embedded into armrest portion 108 of chair 100, for example, in the region identified by reference numeral 103. Such implementations can advantageously allow subject 1 to place an arm, hand, and/or finger(s) proximate the region 103 for the purpose of obtaining one or more physiological measurements. In some implementations, one or more physiological sensors are embedded into the armrest portion 108 underneath a window (for example, an at least partially transparent window) located at region 103. FIG. 1E schematically illustrates an oximetry sensor 109, ECG electrode(s) 111, and temperature sensor(s) 113 which may be incorporated into chair 100 at region 103 in armrest portion 108. The armrest portion 108 can include a flat surface which can include a partially transparent window at region 103 on the armrest portion 108.

In other implementations, the chair 100 can include an oximetry sensor 109, which can be operably positioned within a first armrest portion 108 (for example, where chair 100 includes two armrest portions 108). The oximetry sensor 109 can include at least one emitter configured to emit light into tissue of the subject 1 when in use and at least one detector configured to detect at least a portion of the emitted light after passing through a portion of said tissue when in use. In some implementations, the oximetry sensor 109 includes a plurality of emitters and a plurality of detectors. In some implementations, said at least one emitter and at least one detector are arranged in a reflectance configuration, which can be particularly advantageous when oximetry sensor 109 is positioned underneath a window along armrest portion 108. However, in alternative implementations, chair can include an oximetry sensor that is configured to a transmissive arrangement, for example, where a subject may place a finger within a pocket in or proximate the armrest portion that includes emitter(s) and detector(s) arranged in a transmissive arrangement. In some implementations, chair 100 further includes a second armrest portion 108 connected to the base portion 102 and configured to support another arm of the subject 1 when the subject is seated in the chair and an additional oximetry sensor in such second armrest portion 108. The additional oximetry sensor can be operably positioned within the second armrest portion 108b and can include at least one emitter configured to emit light into tissue of the subject when in use and at least one detector configured to detect at least a portion of the emitted light after passing through a portion of said tissue when in use.

In some implementations, chair 100 includes electrocardiogram (ECG) electrode(s) 111 positioned within a first armrest portion 108 and another ECG electrode(s) 111 operably positioned within a second armrest portion 108. In such implementations, subject 1 can place fingers on each of the ECG electrodes 111, and chair 100 (for example, via a controller of chair 100) can receive one or more signals from the ECG electrodes 111 responsive to cardiac activity of the subject 1. In such implementations, chair 100 can determine physiological status and/or condition of the subject relating to the subject's heart. For example, in some implementations, chair 100 can utilize data derived from such ECG electrodes 111 to generate ECG waveforms on a display of chair 100 (for example, display 110).

In some implementations, chair 100 includes one or more temperature sensors 113 positioned one or more armrest portions 108 of chair 100. Such temperature sensors 113 can be used to determine temperature of the subject 1 (for example, when the subject places a finger, hand, or arm adjacent the temperature sensor 113) and/or to generate signals indicative of the subject's temperature to a processor of chair 100 for temperature determination.

In some implementations, as shown in FIG. 1F, chair 100 includes a leg portion 106′ which can be similar or identical to leg portion 106 except that leg portion 106′ includes one or more cavities configured to receive and surround a portion of one or both legs of the subject 1 when the subject is seated in the chair 100. For example, the leg portion 106′ can partially surround or entirely surround the legs of subject 1 when subject 1 is seated in chair 100. Further, the cavity of leg portion 106′ can include a surface to which the subject 1 can place their feet on top of. In some implementations, portions within such cavities are configured to be inflated and/or vibrated, for example, via utilization of an inflation device (such as inflation device 536) and vibration motor (such as vibration motor 528) of chair 100. In some implementations, portions within such cavities are configured to be inflated and/or vibrated based on one or more physiological parameters determined using one or more physiological measurement devices of chair 100 or in communication with chair 100.

As discussed elsewhere herein, in some implementations chair 100 includes a display, such as display 110 positioned on a back side of back portion 104 (see FIG. 1D). In some implementations, display 110 is located on a side of the back portion 104 that does not face and/or lie adjacent the subject 1 when seated in chair 100. Although display 110 is illustrated in such position in the figures, display 110 can be positioned on a different portion of chair 100, such as armrest portion 108, sidewalls of base portion 102, among other places. Further, although display 110 is illustrated as having a certain size and shape in FIG. 1D, other sizes and/or shapes are possible.

FIG. 1G illustrates a variety of information related to a variety of physiological parameters and/or statuses of a subject that can be displayed on display 110, including but not limited to, oxygen saturation (SpO2), pulse rate (PR), total hemoglobin (SpHb), pleth variability index (PVI); methemoglobin (SpMet), carboxyhemoglobin (SpCO), respiration rate (RRa), blood pressure (systolic and/or diastolic) (for example, noninvasive blood pressure (NIBP)), temperature (in Fahrenheit and/or Celsius), end-tidal cardon dioxide (EtCO₂), EEG data, and/or ECG data. Such data can be obtained from any of the physiological measurement devices disclosed herein, processed and/or not processed, and displayed via display 110, for example, by a processor of chair 100. Display 110 can be similar or identical to any of the display discussed in U.S. Pat. No. 10,383,527 which is incorporated by reference herein in its entirety and forms part of the present disclosure. In some implementations, display 110 is configured to facilitate interaction and/or operation by a user, for example, by touching and/or otherwise interacting with a user interface of display 110. For example, display 110 can include one or more user input devices that can be similar or identical to user input device 532 discussed elsewhere herein. FIG. 1H illustrates additional and/or alternative information that may be present on display 110.

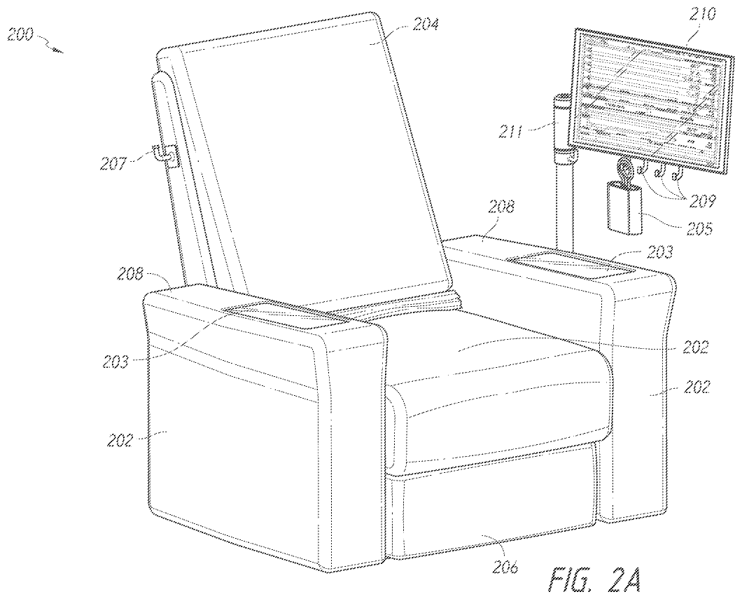

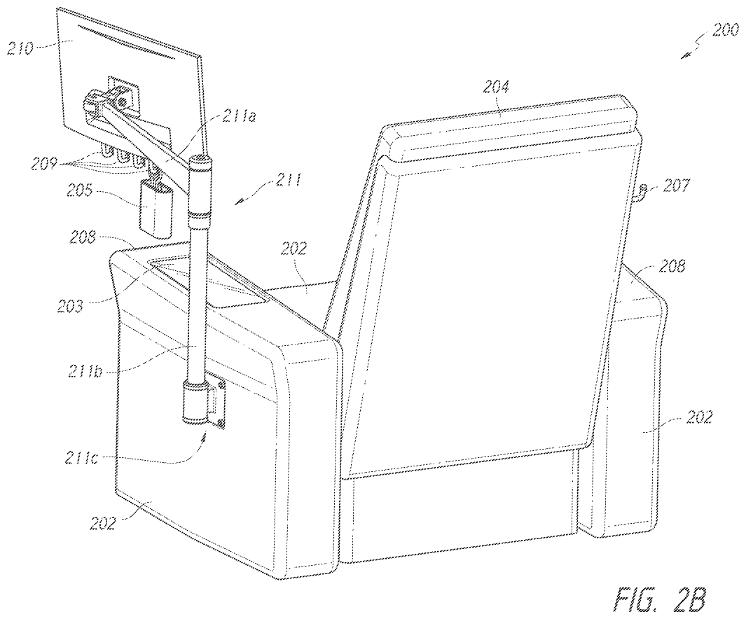

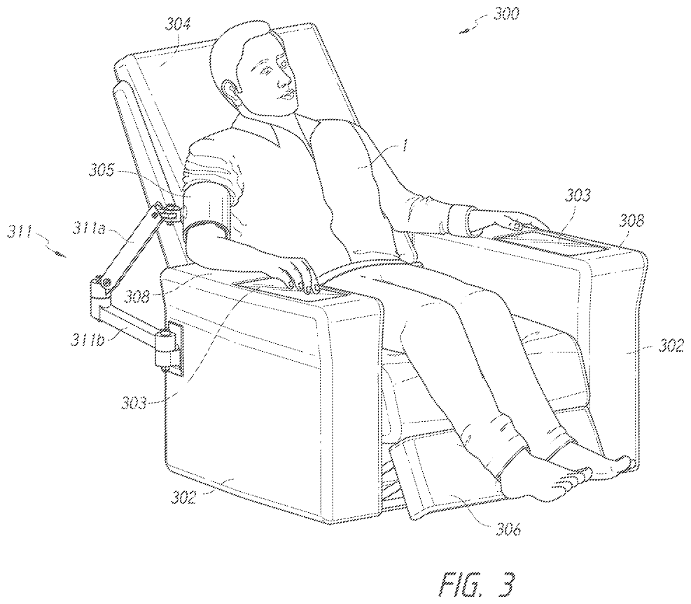

FIGS. 2A-2B illustrate another implementation of a chair 200. Chair 200 can be similar or identical to chair 100 in some or many respects. Chair 200 can include a base portion 202 (which can include a seat and/or one or more sidewalls), back portion 204, leg portion 206, armrest portions 208, each of which can be similar or identical to base portion 102, back portion 104, leg portion 106, and armrest portions 108, respectively. Chair 200 can include hook 207 which can be similar or identical to hook 107. In some implementations, chair 200 includes one or more physiological sensors positioned within armrest portion(s) 208 at regions 203, for example, in a similar or identical manner as that described above with respect to sensor(s) 109, 111, 113, and region 103.

In some implementations, chair 200 does not include a display embedded into a portion of the chair 100 (for example, as shown with respect to display 110), but rather, includes a display 210 that is coupled to a portion of chair 200 but is spaced away from chair 200. Display 210 can be coupled to a portion of chair 200 with a mounting assembly 211 configured to mount the display at a location away from the chair 200. The mounting assembly 211 can include one or more support arms that couple the display 210 to the chair 200. For example, mounting assembly 211 can include support arms 211a, 211b, and, in some implementations, a support mount 211c configured to connect support arm 211b to a portion of the chair 200 (such as a sidewall of base portion 202). While mounting assembly 211 is illustrated as being connected to a sidewall of base portion 202, mounting assembly 211 can be connected to a different portion of chair 200. For example, mounting assembly 211 can be connected to either of two sidewalls of base portion 202 (where base portion 202 has two sidewalls), the back portion 204, or armrest portion 208. In some implementations, support arms 211a, 211b are movable (for example, pivotable) relative to one another so as to allow a position of display 210 to be changed. In some implementations, one or more hooks 209 extend from display 210, for example, to hold cable(s) connected to physiological measurement devices that may be connected and/or connectable to chair 200 and/or to secure a tube connected to an inflatable cuff 205 (which can be similar or identical to cuff 105).

FIG. 3 illustrates another implementation of a chair 300 which can be similar or identical to chair 100. Chair 300 can be identical to chair 100 except that chair 300 includes housing 305 (which can include an inflatable cuff) and mounting assembly 311 instead of cuff 105 and connected tubing discussed above with respect to chair 100. Chair 300 can include a base portion 302 (which can include a seat and/or one or more sidewalls), back portion 304, leg portion 306, armrest portions 308, each of which can be similar or identical to base portion 102, back portion 104, leg portion 106, and armrest portions 108, respectively. In some implementations, chair 300 includes one or more physiological sensors positioned within armrest portion(s) 308 at regions 303, for example, in a similar or identical manner as that described above with respect to sensor(s) 109, 111, 113, and region 103.

Chair 200 can include an inflatable cuff positioned within and coupled to a housing 305. Housing 305 can be tubular and rigid (for example, made of a more rigid material than an inflatable cuff positioned within housing 305). Housing 305 can be sized and/or shaped to receive a portion of an arm of a subject 1, as illustrated in FIG. 3. Further, chair 300 can include a mounting assembly 311 connected to a portion of the chair 300 (for example, a sidewall of base portion 302 of chair 200.

The mounting assembly 311 can include one or more support arms that mount the housing 305 to a portion of the chair 300. In some implementations, mounting assembly 311 includes mounting arms 311a, 311b that can be mounted to and between housing 305 and a portion of the chair 300. In some implementations, mounting arm 311b is mounted to a portion of the chair 300 (for example, a sidewall of chair 300) via a mounting bracket, as shown in FIG. 3. In some implementations, mounting arms 311a, 311b are movable (for example, pivotable) relative to one another so as to allow a position of housing 305 to be changed, which can advantageously allow subject to position housing 305 in a convenient and comfortable manner.

While mounting assembly 311 is illustrated as being connected to a sidewall of base portion 202, mounting assembly 311 can be connected to a different portion of chair 300. For example, mounting assembly 311 can be connected to either of two sidewalls of base portion 302 (where base portion 302 has two sidewalls), the back portion 304, or armrest portion 308. In some implementations, support arms 311a, 311b are movable (for example, pivotable) relative to one another so as to allow a position of housing 305 to be changed.

FIGS. 4A-4D show various illustrative implementations of physiological monitoring chair 400 and various physiological measurement devices which can be connected to (for example, integrated into) chair 400 and/or can interact (for example, wirelessly communicate) with chair 400. Chair 400 can be similar or identical to chairs 100, 200, and 300 in some or many respects. Chair 400 can include a base portion 402 (which can include one or more sidewalls and a seat), a back portion 404, armrest portion(s) 408, and a leg portion (not illustrated), that can be similar or identical in some or many respects to base portion 102, back portion 104, armrest portion(s) 108, and leg portion 106, respectively. In some implementations, chair 300 includes one or more physiological sensors positioned within armrest portion(s) 408 at regions 403, for example, in a similar or identical manner as that described above with respect to sensor(s) 109, 111, 113, and region 103. While not illustrated, chair 400 can include a display such as display 110 embedded into a portion of chair 400 or a display such as display 210 that is mounted to a portion of chair 400 via a mounting assembly (such as mounting assembly 211).

With reference to FIG. 4A, physiological monitoring chair 400 can include one or more of blood pressure monitor 420 (with or without associated cuff 421), ECG device 430 (with or without associated electrodes 432 and/or cables 434), oximetry sensor 440, acoustic sensor 450, and/or EEG sensor 460. In some implementations, chair 400 includes a connection port, such as connection port 412, that can enable removable connection of cables 414a, 414b, 414c, 414d, 414e, and/or 414f that can be connected to and/or can extend from blood pressure monitor 420, ECG device 430, oximetry sensor 440, acoustic sensor 450, and EEG sensor 460. In some implementations, chair 400 provides power to any or all of blood pressure monitor 420, ECG device 430, oximetry sensor 440, acoustic sensor 450, and/or EEG sensor 460 via connection port 412 (via cables 414a, 414b, 414c, 414d, 414e, and/or 414f) and/or allows physiological data to be obtained from any or all of blood pressure monitor 420, ECG device 430, oximetry sensor 440, acoustic sensor 450, and/or EEG sensor 460 to chair 400 (for example, to processor 504 and/or storage device 506 shown in FIG. 5). In some variants, chair 400 includes integral cables similar to any or all of cables 414a, 414b, 414c, 414d, 414e, and/or 414f that are connected to any or all of blood pressure monitor 420, ECG device 430, oximetry sensor 440, acoustic sensor 450, and/or EEG sensor 460, but which do not allow for removable connection. For example, in such variants, such integral cables can extend out from a portion of chair 400 (for example, an electronic module of chair 400). In some variants, chair 400 does not include such a connection port 412.

Oximetry sensor 440, blood pressure monitor 420, ECG device 430, and/or acoustic sensor 450 can be implementations of oximetry sensor 508, blood pressure monitor 510, ECG device 512, and/or acoustic sensor 518 discussed elsewhere herein with reference to FIG. 5. Oximetry sensor 440, blood pressure monitor 420, ECG device 430, and/or acoustic sensor 450 can be similar or identical, respectively, to any of the oximetry sensors, blood pressure monitors, ECG devices, and/or acoustic sensors described in U.S. Publication No. 2020/0329993 (respectively), which is incorporated by reference herein in its entirety and forms part of the present disclosure. EEG sensor 460 can be an implementation of EEG sensor 516 discussed elsewhere herein with reference to FIG. 5. EEG sensor 460 can be similar or identical to any of the sensors described in U.S. Pat. Nos. 8,821,397 and/or 10,154,815, each of which are incorporated by reference herein in their entireties and form part of the present disclosure. For example, sensor 460 can be similar or identical to any of the modular physiological sensors described in U.S. Pat. No. 10,154,815 which can include two regional oximetry sensors configured to attach to an EEG sensor having a stem and left and right branches extending from the stem. In such implementations, EEG sensor 460 can include three cables, two of which can be cables 414e, 414f extending outward from two regional oximetry sensors (having, for example, a rounded end), and the third of which can be cable 414g extending from the branched stem of the EEG sensor 460. Although not shown, cable 414g can be similar to the cables 414a-414f and can connect to connection port 412 in some implementations, or cable 414g can be integral with chair 400 as described above with respect to cables 414a-414f In the illustrated implementation, cables 414e, 414f join (for example at a cable coupler such as cable coupler 477 shown in FIG. 4C) to a cable 414g which can connect to connection port 412.

In some implementations, chair 400 includes one or more hooks configured to hold portions of cables or other objects (such as tubes). For example, chair 400 can include a hook 407 (which can be similar or identical to any of hooks 107, 207, 209.

Connection port 412 can have one or more or a plurality of ports configured to connect to ends of cables (such as any of cables 414a-414h). For example, connection port 412 can include one, two, three, four, five, six, seven, or eight or more ports configured to connect to end of cables.

FIG. 4B illustrates an implementation similar to that shown in FIG. 4A except that EEG sensor 460 is not existent and/or is not attached to the subject 1′, which is why the connection port 412 has two “free” connectors (see FIG. 4B). FIG. 4B also illustrates how a breathing apparatus and/or assembly can be utilized with and/or be part of chair 400. For example, chair 400, including any of the physiological measurement devices discussed herein (among others), can additionally include and/or can be utilized alongside a breathing apparatus and/or assembly including a nasal cannula 470. Nasal cannula 470 can include one or more prongs (for example, two prongs) configured to fit within nostrils of the subject 1′, a manifold, and tubes 470a, 470b (portions of which are illustrated schematically connected to gas source 415) for delivering a gas (for example, a gas comprising oxygen and/or other gaseous compositions) from a gas source 415. In some implementations, both of tubes 470a, 470b deliver gas from gas source 415 to the nostrils, for example, continuously. Nasal cannula 470 can be similar in some or many respects to any of the nasal cannula disclosed in U.S. Pat. No. 10,441,196, which is incorporated by reference herein in its entirety.

Gas source 415 can be, for example, a gas tank). In some implementations, the gas source 415 is integrated into and/or secured to the chair 400. For example, chair 400 can include structure that houses the gas source 415, such as on a back side of the back portion 104 below the display 110 (see FIG. 1D) in implementations of chair 100 including such display. Additionally or alternatively, in some implementations, one or both tubes 470a, 470b extend outwards from portion(s) of the chair 400, for example, portions of the chair 400 that house the gas source 415, and can be connected to the gas source 415. Having the gas source 415 and tubes 470a, 470b on the chair 400 improves management of the tubes 470a, 470b and minimizes the need for such tubes 470a, 470b to span across and/or between chair 400 and conventional locations for gas sources that are positioned away from the place the subject 1′ is seated (for example, in a hospital setting where the gas source is on a wall spaced from the patient bed where the patient is lying). In some variants, one or both of tubes 470a, 470b can be connected to a capnograph (such as capnograph 413) instead of gas source 415 to allow for analysis of characteristics of exhaled breath of subject 1′. One or both of tubes 470a, 470b can be connected to a capnography device such as any of those described in U.S. Pat. No. 10,532,174, which is incorporated by reference herein in its entirety. In some variants, tube 470b delivers gas (for example, comprising oxygen) to the nasal cannula 470 to a first nostril of user 1′ and the tube 470a collects exhaled gas from the other nostril of user 1′ and delivers the exhaled gas to a gas sampling device, for example, in order to determine carbon dioxide content in the exhaled gas. In some implementations, a capnograph can be integrated into the chair 400 and can receive the collected exhaled gas.

Although FIGS. 4A-4B illustrate a single connection port 412, in some implementations, chair 400 can include more than one connection port 412. For example, chair 400 can include, two, three, or four or more connection ports 412. FIG. 4C illustrates an implementation of chair 400 having three connection ports 412, one of which (412a) is connected to a sidewall of chair 400 (proximate armrest portion 408), and the other two of which are connected to side and top portions of back portion 404. Inclusion of a plurality of connection ports 412 can advantageously reduce length requirements for cables connected to sensors secured to the subject and minimize interference of such cables with one another. As shown, chair 400 can include: a connection port 412c positioned on a portion of chair 400 (e.g., a top portion of back portion 404) that is near to a subject's head; a connection port 412c positioned on a portion of chair 400 (e.g., side of back portion 404) that is near to a subject's chest, neck, and/or upper arm; and/or a connection port 412a positioned on a portion of chair 400 that is near to the subject's elbow, lower arm, and/or hand.

Although various implementations of the chairs disclosed herein can include integral cables connected to physiological measurement devices and/or can include removable/connectable cables connected to such devices, any of the chairs disclosed herein can additionally or alternatively be configured to wirelessly communicate with physiological measurement devices. As described elsewhere herein, in some implementations, chair 400 can be configured to wirelessly communicate (for example, via a communication module such as communication module 526 described elsewhere herein) with one or more physiological measurement devices that may be attached to a subject 1′ while the subject is seated in the chair 400 (and/or when the subject is not seated in the chair 400). For example, chair 400 can be configured to communicate with a wearable device 480 that can be secured to a chest or back of the subject 1′ and/or with a sensor assembly 490 that can be secured to an arm (for example, wrist) and/or finger of the subject 1′. Wearable device 480 can be similar or identical to any of the wearable devices disclosed in U.S. Pat. No. 10,383,527 or U.S. Publication No. 2021/0290072, which are incorporated by reference herein in their entireties and form part of the present disclosure. Sensor assembly 490 can be similar or identical to any of the sensor assemblies disclosed in U.S. Publication No. 2020/0138288, which is incorporated by reference herein in its entirety and form part of the present disclosure.

Although not illustrated in the figures, in some implementations, chair 100 includes and/or is configured to communicate with an auricular device configured to attach to a subject's ear. Such auricular device can be similar or identical to any of the auricular devices described in U.S. Pat. No. 8,588,880, which is incorporated by reference herein in its entirety and forms part of the present disclosure.

FIG. 5 illustrates a schematic diagram of a physiological monitoring chair 500. Any of the features discussed with respect to FIG. 5 can be incorporated into any of the chairs disclosed herein (such as chair 100, 200, 300, and 400).

Chair 500 can include a controller 502, a processor 504 (or one or more or a plurality of processors), and/or a storage device 506. [

The processor 504 can be configured, among other things, to process data, execute instructions to perform one or more functions, and/or control the operation of the chair 500. For example, the processor 504 can process physiological data obtained from one or more physiological measurement devices of and/or in communication with chair 500 (such as any of those discussed herein) and can execute instructions to perform functions related to storing and/or transmitting such physiological data. For example, the processor 504 can process data received from one or more physiological measurement devices of and/or in communication with chair 500, such as any or all of oximetry sensor 508, blood pressure monitor 510, ECG sensor 512, capnograph 514, EEG sensor 516, acoustic sensor 518, temperature sensor 520, and/or any other sensor(s) 522 of chair 500, each of which are discussed elsewhere herein. The processor 504 can execute instructions to perform functions related to storing and/or transmitting any or all of such received data.

The storage device 506 can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed physiological data or other types of data (for example, motion and/or location data) obtained from one or more physiological measurement devices of chair 100 (such as any of those discussed herein).

In some implementations, chair 500 includes an information element 530. The information element 530 can be a memory storage element that stores, in non-volatile memory, information used to help maintain a standard of quality associated with chair 500. Illustratively, the information element 530 can store information regarding whether chair 500 (for example, any of one or more physiological measurement devices of chair 500) has been previously activated and whether the chair 500 (for example, any of one or more physiological measurement devices of chair 500) has been previously operational for a prolonged period of time, such as, for example, one, two, three, four, five, six, seven, or eight or more hours. The information stored in the information element 530 can be used to help detect improper use of the chair 500, for example.

In some implementations, the chair 500 includes a user input device 532. User input device 532 can allow a user (for example, a subject seated in chair 500 and/or a caregiver) to interact with chair 500, for example, to instruct chair 500 to carry out one or more actions. Such actions can include without limitation: causing one or more physiological measurement devices to turn on and/or take measurements; causing vibration of portions of chair 500; transmitting data or notifications to a caregiver; changing a position of the chair 500 (for example, upright, reclined, flat); and/or other actions. The user input device 532 can be implemented as a touch-sensitive device and/or a movable device (for example, a button), for example. In some implementations, the user input device 532 can be integrated into a portion of chair 500 for example, an armrest portion of chair 500 (which can be similar or identical to any of the armrest portions discussed herein).

The communication module 526 can facilitate communication (via wires and/or wireless connection) between the chair 500 (and/or components thereof) and separate devices, such as separate monitoring and/or mobile devices. For example, the communication module 526 can be configured to allow the chair 500 to wirelessly communicate with other devices, systems, and/or networks over any of a variety of communication protocols. The communication module 526 can be configured to use any of a variety of wireless communication protocols, such as Wi-Fi (802.11x), Bluetooth®, ZigBee®, Zwave®, cellular telephony, infrared, near-field communications (NFC), RFID, satellite transmission, proprietary protocols, combinations of the same, and the like. The communication module 526 can allow data and/or instructions to be transmitted and/or received to and/or from the chair 100 and separate computing devices. The communication module 526 can be configured to transmit (for example, wirelessly) processed and/or unprocessed physiological or other information to a separate computing devices, which can include, among others, one or more computing devices at a caregiver station in a hospital, a mobile device (for example, an iOS or Android enabled smartphone, tablet, laptop), a desktop computer, a server or other computing or processing device for display and/or further processing, among other things. Such separate computing devices can be configured to store and/or further process the received physiological and/or other information, to display information indicative of or derived from the received information, and/or to transmit information—including displays, alarms, alerts, and notifications—to various other types of computing devices and/or systems that may be associated with a hospital, a caregiver (for example, a primary care provider), and/or a user (for example, an employer, a school, friends, family) that have permission to access the subject's data. As another example, the communication module 526 of the chair 500 can be configured to wirelessly transmit processed and/or unprocessed obtained physiological information and/or other information (for example, motion and/or location data) to a mobile phone which can include one or more hardware processors configured to execute an application that generates a graphical user interface displaying information representative of the processed or unprocessed physiological and/or other information obtained from the chair 500. The communication module 526 can be and/or include a wireless transceiver. In some implementations, the communication module 526 can wirelessly communicate with (for example, instruct, obtain physiological data from, among other things) one or more physiological measurement devices that are attached to a subject sitting in chair 400, for example, as illustrated and described with respect to FIG. 4D.

With continued reference to FIG. 5, chair 500 can include a power source 524. Such power source 524 can be, for example, a battery. Such battery can be rechargeable or non-rechargeable. The power source 524 can provide power for the hardware components of the chair 500 described herein. The power source 524 can be, for example, a lithium battery. Additionally or alternatively, the chair 500 can be configured to obtain power from a power source that is external to the chair 500. For example, chair 500 can include or can be configured to connect to a cable which can itself connect to an external power source to provide power to the chair 500. In some implementations, the chair 500 does not include power source 524.

In some implementations, chair 500 includes a vibration motor 528. Vibration motor 528 can be configured to vibrate one or more portions of chair 500, which in turn can vibrate one or more portions of a subject's body when the subject is seated in the chair 500. In some implementations, to vibrate portions of the chair 500, the vibration motor 528 can rotate unbalanced and weighted gears or wheels in which the rotation or movement of said gears or wheels causes a vibration motion. In some implementations, vibration motor 528 is connected to a series or combination of rollers located throughout the chair 500. The rollers can be attached to a structure of frames within the chair 500, and such frames providing a pattern or pathways for the rollers, such as in a lateral or a vertical direction. Vibration motor 528 can further be connected to mechanical arms that offer a greater degree of freedom regarding the pattern of movements and may have a greater freedom of movement which is limited to only lateral or vertical patterns.

Vibration motor 528 can be configured to vibrate various portions of chair 500. Any of the chairs disclosed herein (such as chairs 100, 200, 300, 400) can be implementations of chair 500. Where chair 500 includes a base portion, back portion, armrest portion, and/or leg portion (each of which can be similar or identical to base portion 102, back portion 104, armrest portion 108, and leg portion 106, respectively), vibration motor 528 can be configured to vibrate any of such base portion, back portion, armrest portion, and/or leg portions of chair 500. Controller 502 can be in communication with vibration motor 528 and can be configured to instruct vibration motor 528 to cause any of such above-described vibration. As described in more detail below, in some implementations, chair 500 (for example, via controller 502) is configured to cause vibration of one or more portions of a subject's body (when the subject is seated in chair 500) responsive to one or more physiological parameters determined by chair 500 and/or by one or more physiological measurement devices of chair 500 and/or in communication with chair 500. In some implementations, chair 500 includes more than one vibration motor 528, for example, two, three, four, five, six, seven, eight, nine, or ten or more vibration motors 528. Vibration motor(s) 528 can be positioned within various portions of chair 500, for example, within a base portion, back portion, leg portion, and/or armrest portion of chair 500 (each of which can be similar or identical to base portion 102, back portion 104, armrest portion 108, and leg portion 106, respectively).

In some implementations, chair 500 is configured to cause vibration of one or more portions of a subject's body (when the subject is seated in chair 500) responsive to one or more physiological parameters determined by chair 500 and/or by one or more physiological measurement devices of chair 500 and/or in communication with chair 500. Controller 502 can be in communication with one or more physiological measurement devices (such as any of those mentioned herein) and can instruct the vibration motor 528 to cause vibration, cease vibrating, and/or instruct the vibration motor 528 to alter a characteristic of vibration (for example, increase/reduce vibration rate, change vibration pattern, etc.) responsive to one or more determined physiological parameters. Such action by controller 502 can dynamically track with physiological parameter determination over time, for example. As an example, in some implementations, controller 502 can provide instructions to vibration motor 528 (such as those discussed above) responsive to a condition of a subject in the chair 500. For example, if one or more physiological parameters determined using one or more physiological measurement devices of the chair 500 and/or otherwise in communication with chair 500 are indicative of hypoxemia (low blood oxygen) when the subject is sleeping in the chair 500, the controller 502 can instruct the vibration motor 528 to vibrate to cause the subject to wake up in an attempt to restore proper breathing and/or safe blood oxygen levels. As another example, if one or more physiological parameters determined using one or more physiological measurement devices of the chair 500 and/or otherwise in communication with chair 500 are indicative of edema (swelling caused by excess fluid trapped in body tissue), the controller 502 can instruct the vibration motor 528 to cause vibration of the subject's lower torso, legs, back, and/or any other portion of the subject's body.

In some implementations, based on a first physiological parameter measurement from a physiological measurement device (such as any of those mentioned herein), controller 502 instructs the physiological measurement device to take a second physiological parameter measurement. For example, based on a comparison of such first physiological parameter measurement to a threshold (for example, falls below or above such threshold), controller 502 instructs the physiological measurement device to take a second physiological parameter measurement.

As discussed elsewhere herein, chair 500 can be in communication with one or more separate computing devices and can transmit physiological data to such one or more separate computing devices. For example, chair 500 can be in communication with a separate computing device at a caregiver's station in a hospital and/or a mobile phone of the subject sitting in the chair 500 or another person. In some implementations, chair 500 (for example, via communication module 526) transmits one or more physiological parameter measurements to a separate computing device, receives one or more instructions from the separate computing device, and instructs one or more physiological measurement devices to perform one or more additional physiological parameter measurements. This can advantageously allow such separate computing device to operate, at least in part, the chair 500 from a remote location in order to obtain more physiological information regarding the subject seated in chair 500. For example, chair 500 can transmit physiological parameter measurement(s) determined using one or more of oximetry sensor 508, blood pressure monitor 510, ECG sensor 512, temperature sensor 520 (among others) to a separate computing device, receive instruction(s) from the separate computing device, and instruct one or more of oximetry sensor 508 blood pressure monitor 510, ECG sensor 512, temperature sensor 520 (among others) to perform one or more additional physiological parameter measurements. As another example, chair 500 can transmit a first physiological parameter measurement determined using a first physiological measurement device to a separate computing device, receive an instruction from the separate computing device to determine at least a second physiological parameter measurement, and instruct the first physiological measurement device and/or a second physiological measurement device to perform the second physiological parameter measurement. For example, chair 500 can transmit a physiological parameter measurement (for example, oxygen saturation and/or pulse rate) determined using oximetry sensor 508 to a separate computing device, receive an instruction from the separate computing device to perform another physiological parameter measurement (such as blood pressure, temperature, EEG waveform, etc.), and then instruct a different physiological measurement device (for example, blood pressure monitor 510, temperature sensor 520, and/or ECG sensor 512) to perform physiological parameter measurement. Such examples are just some of many various possibilities in which chair 500 can interact with a separate computing device to allow for remote operation and determination of physiological parameters of a subject seated in chair 500.

In some implementations, chair 500 includes an oximetry sensor 508. Oximetry sensor 508 can be connected to a portion of the chair 500 (for example, an electronic module of the chair 500) for example, via a cable. The oximetry sensor 508 (which may also be referred to as an “optical sensor”) can include one or more emitters and one or more detectors for obtaining physiological information indicative of one or more blood parameters of a subject. These parameters can include various blood analytes such as oxygen, carbon monoxide, methemoglobin, total hemoglobin, glucose, proteins, glucose, lipids, a percentage thereof (for example, concentration or saturation), and the like. The oximetry sensor 107 can also be used to obtain a photoplethysmograph, a measure of plethysmograph variability, pulse rate, a measure of blood perfusion, and the like. Information such as oxygen saturation (SpO₂), pulse rate, a plethysmograph waveform, respiratory effort index (REI), acoustic respiration rate (RRa), EEG, ECG, pulse arrival time (PAT), perfusion index (PI), pleth variability index (PVI), methemoglobin (MetHb), carboxyhemoglobin (CoHb), total hemoglobin (tHb), glucose, can be obtained from oximetry sensor 107 and data related to such information can be processed and/or transmitted by the chair 500 (for example, via communication module 526) to a separate computing device (such as a computing device at a caregiver's workstation or mobile phone).

In some implementations, chair 500 includes a blood pressure monitor 510. Blood pressure monitor 510 can be connected to a portion of the chair 500 (for example, an electronic module of the chair 500) for example, via a cable. Blood pressure monitor 510 can be configured to measure blood pressure of a subject, for example, when the subject is seated in chair 500. Blood pressure monitor 510 can be similar or identical to any of the blood pressure monitors described in U.S. Publication No. 2020/0329983, which is incorporated by reference herein in its entirety and forms part of the present disclosure. In some implementations, chair 500 includes and/or is coupled to a cuff, which can be similar or identical to cuff 105 and connected tubing. In such implementations, chair 500 can include an inflation device 536 to control the inflation of such cuff. The inflation device 536 can be configured to connect to a portion of the chair 500 and then to such cuff via a tube or directly to such cuff. In some instances, the inflation device can be integrated into the chair 500. In some implementations, inflation device 536 can be utilized to inflate portions of chair 500, for example, where chair 500 includes a leg portion that has cavities configured to receive legs of a subject, such inflation device 536 can inflate portions of the chair 500 within such cavities and around the subject's legs (or portions thereof). Such inflation of such cavities can be responsive to and/or based on physiological parameter(s) of the subject. For example, if one or more physiological parameters are indicative of edema (for example, in portions of the subject's leg and/or foot), the controller 502 can cause inflation device 536 to inflate portions of the chair 500 within such cavities to mitigate such edema.

In some implementations, chair 500 includes an electrocardiogram (ECG) sensor 512 (which may also be referred to as an “ECG device”). In some implementations, ECG device 512 includes ECG electrodes embedded into portions of the chair 500, such as discussed with reference to ECG electrode(s) 111 and chair 100. In some implementations, ECG device 512 is connected to a portion of the chair 500 (for example, an electronic module of the chair 500) for example, via a cable. ECG device 512 can be used to measure electrical activity of a heart of a subject, for example, when the subject is seated in chair 512, and can include one or more electrodes. ECG device 111 can be similar or identical to any of the ECG devices described in U.S. Publication No. 2020/0329983, which is incorporated by reference herein in its entirety and forms part of the present disclosure.

In some implementations, chair 500 includes a device that measures aspects of a subject's respiratory system and/or health. For example, the chair 500 can include respiratory gas measuring devices described in U.S. Pat. No. 10,532,174, which is incorporated by reference herein in its entirety and forms part of the present disclosure. In some implementations, chair 100 includes one or more devices that breathing tubes and/or a cannula that facilitate delivery and/or collection of gases to and/or from the subject and which can be coupled with a capnograph 514, such as the EMMA® Capnograph manufactured and sold by Masimo Corporation. This can advantageously enable measurements of physiological parameters such as end tidal respiratory gases including oxygen (O₂), carbon dioxide (CO₂), and nitrous oxide (N₂O), among others, as well as patient respiratory rate, among others.

In some implementations, chair 500 includes an electroencephalogram (EEG) sensor 516. EEG sensor 516 can be connected to a portion of the chair 500 (for example, an electronic module of the chair 100) for example, via one or more cables. EEG sensor 516 can be used to measure electrical activity of a brain of a subject, for example, when the subject is seated in chair 500, and can include one or more electrodes. EEG sensor 516 can be used to determine, among other things, state of consciousness of the brain. EEG sensor 516 can be similar or identical to any of the EEG sensors described in U.S. Pat. Nos. 8,821,397 and/or 10,154,815, each of which are incorporated by reference herein in their entireties and form part of the present disclosure.

In some implementations, chair 500 includes an acoustic sensor 518. Acoustic sensor 518 can be connected to a portion of the chair 500 (for example, an electronic module of the chair 500) for example, via a cable. The acoustic sensor 518 (also referred to as an “acoustic respiratory sensor” or “respiratory sensor”) can comprise an acoustic transducer, such as a piezoelectric element. The acoustic sensor 518 can detect respiratory and other biological sounds of a subject (for example, when seated in chair 500) and provide signals reflecting these sounds to chair 500 (for example, to processor 504). The acoustic sensor 518 can be a piezoelectric sensor or the like that obtains physiological information reflective of one or more respiratory parameters of the subject. These parameters can include, for example, respiratory rate, inspiratory time, expiratory time, inspiration-to-expiration ratio, inspiratory flow, expiratory flow, tidal volume, minute volume, apnea duration, breath sounds, rales, rhonchi, stridor, and changes in breath sounds such as decreased volume or change in airflow. In addition, in some cases the acoustic sensor 518 can measure other physiological sounds such as heart rate (e.g., to help with probe-off detection), heart sounds (for example, S1, S2, S3, S4, and murmurs), and changes in heart sounds such as normal to murmur or split heart sounds indicating fluid overload. In some implementations, the chair 500 includes two acoustic sensors 518, one of which is placed over the chest of the subject for heart sound detection and the other of which is placed in a different location (for example, a neck of the subject). Acoustic sensor 518 can be implemented as illustrated and described with respect to acoustic sensor 450.

In some implementations, the acoustic sensor 518 can be used to generate an exciter waveform that can be detected by the optical sensor 508 at the fingertip and/or ear of subject, by ECG device 512, or by another acoustic sensor. The velocity of the exciter waveform can be calculated by processor 504. From this velocity, the processor 504 can derive a blood pressure measurement or blood pressure estimate. The processor 504 can output the blood pressure measurement for display. The processor 504 can also use the blood pressure measurement to determine whether to trigger a blood pressure cuff, for example that is coupled to blood pressure monitor 510.

In some implementations, chair 500 includes one or more temperature sensors 520 configured to determine temperature values of the subject (for example, when seated in chair 500) and/or that are configured to generate and/or transmit signal(s) based on detected thermal energy of the user to processor 504 for determination of temperature value(s). In some implementations, temperature sensor(s) 520 are contained in a wearable device that can be connected to a portion of the chair 500 (for example, an electronic module of the chair 500) for example, via a cable. Such wearable device can be similar or identical to any of the wearable devices described in U.S. Publication No. 2021/0290072 and temperature sensor(s) 520 can be configured to determine body temperature in a manner such as described in U.S. Publication No. 2021/0290072, which is incorporated by reference herein in its entirety and forms part of the present disclosure.

In some implementations, chair 500 includes one or more other sensor(s) 522. Such other sensor(s) 522 can comprise, for example, one or more sensors that obtain information of the subject (for example, when seated in chair 500) related to position, orientation, and/or movement. For example, such other sensor(s) 522 can comprise an accelerometer, a gyroscope, and/or a magnetometer. Such other sensor(s) 522 can comprise a wearable device that includes an accelerometer and/or a gyroscope similar or identical to any of the wireless sensors described in U.S. Pat. No. 10,383,527, which is incorporated by reference herein in its entirety and forms part of the present disclosure. Such other sensor(s) 522 can be connected to a portion of the chair 500 (for example, an electronic module of the chair 500) for example, via a cable.

In some implementations, chair 500 (or any of the other chairs disclosed herein, such as chair 100, 200, 300, 400) can detect whether subject is seated and/or laying in chair 500 and, responsive to such detection: instruct one or more physiological measurement devices connected to the chair 500 (via wireless and/or wired means) to turn on and/or begin collecting physiological data; display (for example, on a display of the chair or connected to a portion of the chair) a notification (e.g., an alert) that the subject is seated and/or laying in the chair; transmit one or more signals to an external device to notify the external device that the subject is seated and/or laying in the chair; turn off and/or instruct one or more physiological measurement devices connected to the chair (via wireless and/or wired means) when the subject is not seated and/or laying in the chair; cause an audible alert to be emitted (for example, from a speaker of the chair) when the subject is not seated and/or laying in the chair.

With continued reference to FIG. 5, chair 500 can include a display 534 that can similar or identical to any of the displays disclosed herein (such as display 110).

Although the figures illustrate example configurations of chairs, for example, having base portions, back portions, leg portions, and/or armrest portions, the illustrated configurations are not intended to be limiting. The present disclosure contemplates chairs that can be of a variety of types and/or configurations.

### Additional Considerations and Terminology

Although this invention has been disclosed in the context of certain preferred embodiments, it should be understood that certain advantages, features and aspects of the systems, devices, and methods may be realized in a variety of other embodiments. Additionally, it is contemplated that various aspects and features described herein can be practiced separately, combined together, or substituted for one another, and that a variety of combination and subcombinations of the features and aspects can be made and still fall within the scope of the invention. Furthermore, the systems and devices described above need not include all of the modules and functions described in the preferred embodiments.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

Although certain embodiments and examples have been described herein, it will be understood by those skilled in the art that many aspects of the systems and devices shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (for example, not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

Various illustrative logical blocks, modules, routines, and algorithm steps that may be described in connection with the disclosure herein can be implemented as electronic hardware (e.g., ASICs or FPGA devices), computer software that runs on general purpose computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the disclosure herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor. A processor can include electrical circuitry configured to process computer-executable instructions. A processor can include an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1.-71. (canceled)

72. A physiological monitoring chair for monitoring one or more physiological parameters of a subject, the physiological monitoring chair comprising:
a base portion configured to rest atop a surface and support a weight of the subject;
a back portion connected to the base portion and configured to at least partially support a back of the subject when the subject is seated in the physiological monitoring chair;
a leg portion connected to the base portion and configured to be positioned adjacent to legs of the subject when the subject is seated in the physiological monitoring chair;
at least one vibration motor;
one or more struts connected to said leg portion and said back portion;
a strut motor configured to move said one or more struts; and
a controller in communication with said at least one vibration motor and said strut motor, wherein the controller is configured to:
receive, from at least one physiological measurement device, a plurality of physiological parameters of the subject;
instruct the at least one vibration motor to cause vibration of at least a portion of the physiological monitoring chair when a first value of a first one of said plurality of physiological parameters exceeds a first threshold;
instruct the at least one vibration motor to stop vibration of said portion of the physiological monitoring chair when a second value of said first one of said plurality of physiological parameters is less than said first threshold;
instruct the strut motor to move said one or more struts to cause the physiological monitoring chair to transition from a first configuration to a second configuration when at least one of:
the first value of the first one of said plurality of physiological parameters exceeds said first threshold; and
a value of a second one of said plurality of physiological parameters exceeds a second threshold;
wherein, when the physiological monitoring chair is in said first configuration, at least one of said back portion and said leg portion is oriented non-parallel with respect to said surface, and wherein when the physiological monitoring chair is in said second configuration, said back portion and said leg portion are oriented substantially parallel with respect to said surface.

73. The physiological monitoring chair of claim 72, further comprising said at least one physiological measurement device, wherein said at least one physiological measurement device is connected to a portion of said physiological monitoring chair via a cable, and wherein said physiological monitoring chair is configured to provide power to said at least one physiological measurement device.

74. The physiological monitoring chair of claim 72, further comprising a display configured to display information relating to said plurality of physiological parameters of the subject.

75. The physiological monitoring chair of claim 72, wherein the controller is configured to wirelessly receive physiological information from the at least one physiological measurement device.

76. The physiological monitoring chair of claim 72, wherein the leg portion includes cavities, each of said cavities configured to receive and at least partially surround a leg of the subject, and wherein the controller is further configured to inflate a portion of each cavity when said first value of the first one of said plurality of physiological parameters exceeds said first threshold.

77. The physiological monitoring chair of claim 72, wherein the controller is configured to instruct the at least one vibration motor to cause vibration of the leg portion of the physiological monitoring chair when said first value exceeds said first threshold.

78. A physiological monitoring chair for monitoring one or more physiological parameters of a subject, the physiological monitoring chair comprising:
a base portion configured to rest atop a surface and support a weight of the subject;
a back portion connected to the base portion and configured to at least partially support a back of the subject when the subject is seated in the physiological monitoring chair;
a leg portion connected to the base portion and configured to be positioned adjacent to legs of the subject when the subject is seated in the physiological monitoring chair;
at least one vibration motor; and
a controller in communication with the at least one vibration motor, wherein the controller is configured to:
receive one or more physiological parameters of the subject from at least one physiological measurement device; and
instruct the at least one vibration motor to cause vibration of at least a portion of the physiological monitoring chair based on said one or more physiological parameters of the subject.

79. The physiological monitoring chair of claim 78, wherein the controller is further configured to instruct the at least one vibration motor to cause vibration of said portion of the physiological monitoring chair when a first value of said one or more physiological parameters exceeds a first threshold.

80. The physiological monitoring chair of claim 79, wherein the controller is further configured to instruct the at least one vibration motor to stop vibration of said portion of the physiological monitoring chair when a second value of said one or more physiological parameters is less than said first threshold.

81. The physiological monitoring chair of claim 78, wherein:
the controller is further configured to cause the physiological monitoring chair to transition from a first configuration to a second configuration when a first value of said one or more physiological parameters exceeds a first threshold;
when the physiological monitoring chair is in said first configuration, at least one of said back portion and said leg portion is oriented non-parallel with respect to said surface; and
when the physiological monitoring chair is in said second configuration, said back portion and said leg portion are oriented substantially parallel with respect to said surface.

82. The physiological monitoring chair of claim 78, further comprising said at least one physiological measurement device.

83. The physiological monitoring chair of claim 82, wherein said at least one physiological measurement device comprises at least one of an oximetry sensor and a blood pressure monitor.

84. The physiological monitoring chair of claim 83, further comprising a armrest portion connected to the base portion and configured to support an arm of the subject when the subject is seated in the physiological monitoring chair, wherein said at least one physiological measurement device comprises an oximetry sensor integrated into the armrest portion.

85. The physiological monitoring chair of claim 78, further comprising a display configured to display information relating to said one or more physiological parameters of the subject.

86. The physiological monitoring chair of claim 85, wherein said display is located on a side of the back portion that does not face toward the subject when the subject is seated in the physiological monitoring chair.

87. The physiological monitoring chair of claim 85, wherein said display is connected to a mounting assembly connected to the base portion and configured to mount the display at a location away from the base portion.

88. A physiological monitoring chair for monitoring one or more physiological parameters of a subject, the physiological monitoring chair comprising:
a base portion configured to rest atop a surface and support a weight of the subject;
a back portion connected to the base portion and configured to at least partially support a back of the subject when the subject is seated in the physiological monitoring chair;
a leg portion connected to the base portion and configured to be positioned adjacent to legs of the subject when the subject is seated in the physiological monitoring chair; and
a controller configured to:
receive one or more physiological parameters of the subject from at least one physiological measurement device; and
cause the physiological monitoring chair to transition from a first configuration to a second configuration when a first value of said one or more physiological parameters exceeds a first threshold;
wherein at least one of the back portion and leg portion is oriented at a different angle relative to said surface when the physiological monitoring chair is in said first configuration than when the physiological monitoring chair is in said second configuration.

89. The physiological monitoring chair of claim 88, further comprising a motor and one or more struts connected to said motor and to said back portion and said leg portion, wherein said controller is configured to cause the physiological monitoring chair to transition from the first configuration to the second configuration by instructing the motor to move the one or more struts.

90. The physiological monitoring chair of claim 88, wherein said one or more physiological parameters comprises blood pressure of the subject.

91. The physiological monitoring chair of claim 88, wherein when the physiological monitoring chair is in said first configuration, at least one of said back portion and said leg portion is oriented non-parallel with respect to said surface, and wherein when the physiological monitoring chair is in said second configuration, said back portion and said leg portion are oriented substantially parallel with respect to said surface.
